(19) United States
(12) Patent Application Publication
(10) Pub. No.: US 2023/0363846 A1
(43) Pub. Date: Nov. 16, 2023

(54) DISPOSABLE SURGICAL TUBE AND CORD CONTAINMENT DRAPE THEREWITH, AND METHODS OF CONSTRUCTION THEREOF

(71) Applicant: Domico Med-Device, LLC, Fenton, MI (US)

(72) Inventors: Michael W. Czop, Fenton, MI (US); Jaime E. Morrell, Grand Blanc, MI (US); Samba Toure, Grand Blanc, MI (US)

(21) Appl. No.: 18/197,101

(22) Filed: May 14, 2023

Related U.S. Application Data
(60) Provisional application No. 63/452,425, filed on Mar. 15, 2023.
(60) Provisional application No. 63/341,643, filed on May 13, 2022.

(51) Int. Cl. A61B 46/10

(52) U.S. Cl. CPC A61B 46/10

(57) ABSTRACT

A disposable surgical drape facilitates surgical field organization, and retains and shields at least one medical tube and/or cord against contamination. The surgical drape includes a flexible wall having opposite edges extending lengthwise between opposite ends and a fixation member extending adjacent one of the opposite edges. The drape includes a wall having opposite edges extending between opposite ends. The wall has an open state, wherein tubes and/or cords can be disposed into the drape, and a closed state, wherein the tubes and/or cords are protected by the drape. When in the open state, one of the opposite edges can be folded toward the other of the opposite sides to bring the wall to the closed state to protect the tubes and/or cords in an inner cavity bounded by the flexible wall.

### CROSS-REFERENCE TO RELATED APPLICATIONS

This application claims the benefit of U.S. Provisional Application Ser. No. 63/452,425, filed Mar. 15, 2023, and the benefit of U.S. Provisional Application Ser. No. 63/341,643, filed May 13, 2022, which are both incorporated herein by reference in their entirety.

### BACKGROUND

### 1. Technical Field

This invention relates generally to tube and cord management and protection apparatus, and more particularly to disposable tube and cord containment apparatus for protecting and organizing tubes and cords in an operating room, including tubes and cords draped over a patient laying on a patient support table, and patient drape therewith.

### 2. Related Art

The need to provide and maintain a clean, contamination free, and often sterile environment in a medical arena is well established. Patient contamination, such as from patient fluids, including blood and urine, for example, is directly associated with the known risk of infection that can be caused by bacteria from such contamination in the medical arena. As such, it is known that in order to minimize the risk of infection during a medical procedure, it is necessary to prevent the transfer of bacteria from contamination within the medical arena. Although the risks of contamination and infection during medical procedures in an operating room are recognized, it remains a goal to prevent the onset thereof. Particular challenges include preventing the contamination of surfaces within the operating room, including fluid and gas conveying tubes, electrical cords, and the like, such as those draped over a patient and surgical equipment stand. Infections have been estimated to affect upwards of 2 million patients or more annually. Aside from the complications brought about by infections, the infections create an economic burden on hospitals. For example, some studies have estimated that each bloodstream infection in a patient results in an average cost of about $27,000 to the hospital. Accordingly, advances are needed to reduce the potential of contamination within the medical arena and infection resulting therefrom.

Additionally, tubes and/or cords that become contaminated within a sterile zone of the operating room, typically defined above a patient support surface, such as those draped on an instrument stand, known as a Mayo stand, and/or over a patient during the medical procedure, must be immediately replaced, which generally causes unwanted, costly delay of the medical procedure. Further complicating matters is when contaminated tubes and/or cords are entangled with one another or with other non-contaminated tubes and/or cords. Having to replace an entangled tube and/or cord is generally time consuming and complicated, and can often lead to unintended contamination of other tubes, cords, or surgical tools and equipment.

There is no standard methodology for organizing tubes and/or cords and shielding tubes and/or cords against contamination and against the transfer of contamination. One known methodology includes laying a cloth or cloths over tubes and cords, but this methodology does not prevent contamination from soaking through the cloth, and does nothing to maintain the tubes and cords in organized arrangement with one another. Further yet, the cloths can prove problematic by obstructing visualization of the tubes and cords. Generally, it is desirable to be able to visualize fluid flowing through a tube to ensure the tube is not clogged, and it is further desirable to be able readily identify a cord connected to a specific instrument. Another current method includes using forceps and clamps to clamp items directly to the patient drape, however, this can result in damage, e.g. tearing, to the patient drape, which compromises sterility. Also, having to individually clamp items takes a excessive time, and thus, is inefficient. Further yet, unwanted constriction of a tube or damage to and electrical wire can result if clamped too tightly.

Accordingly, a need exists for an apparatus and method for organizing tubes and/or cables, for addressing at least those problems discussed above, including shielding the tubes and/or cables against contamination, allowing ease of set-up and replacement of tubes and/or cords, while allowing visualization of the uncompromised tubes and/or cords during medical procedure, preventing damage to the surgical drape, while being economical in manufacture and in use, and while being user friendly.

### SUMMARY OF THE INVENTION

This section provides a general summary of the disclosure, and is not intended to be a comprehensive and exhaustive listing of all of its features or its full scope.

It is an object of the present disclosure to provide a disposable surgical tube and/or cord containment drape for shielding at least one tube and/or cord against contamination.

It is a further object of the present disclosure to provide a disposable surgical tube and/or cord containment drape for bundling a plurality of the tube and/or cord with one another is provided, thereby allowing select tubes and/or cords to be grouped, as desired.

It is a further object of the present disclosure to provide a disposable surgical tube and/or cord containment drape that allows quick and easy identification of and access to one or more tubes and/or cords during a surgical procedure to allow quick and easy replacement of the one or more tubes and/or cords, as needed, if the one or more tubes and/or cords becomes contaminated, clogged, or damaged.

It is a further object of the present disclosure to provide a disposable surgical drape having at least one surgical tube and/or cord management drape for shielding at least one tube and/or cord against contamination.

It is a further object of the present disclosure to provide the disposable surgical drape, having at least one surgical tube and/or cord management drape for shielding at least one tube and/or cord against contamination, configured to drape a patient on a surgical table.

It is a further object of the present disclosure to provide the disposable surgical drape, having at least one surgical tube and/or cord management drape for shielding at least one tube and/or cord against contamination, configured to drape a surgical instrument stand, such as a Mayo stand.

In accordance with these and other objects, as will be appreciated by a person possessing ordinary skill in the art in view of the disclosure herein, a disposable surgical tube and/or cord containment drape, referred to hereafter as drape, includes a flexible wall having opposite edges extending lengthwise between opposite ends. The flexible wall has an open state, wherein one or more tubes and/or cords can be easily disposed into the drape, and a closed state, wherein the one or more tubes and/or cords are contained and protected in the drape. While the flexible wall is in the open state, upon disposing the desire tube(s) and/or cord(s) along the flexible wall of the drape, one of the opposite edges is configured to be folded about the at least one tube and/or cord toward the other of the opposite sides to bring the flexible wall to the closed state to contain and shield the at least one tube and/or cord in an inner cavity bounded by an inner surface of the flexible wall. At least one closure member is fixed to the flexible wall, with the at least one closure member being configured to releasably fix the flexible wall in the closed state.

In accordance with another aspect of the disclosure, the at least one closure member can include a first fastener portion and a second fastener portion, with the first fastener portion being configured for releasably fixed attachment to the second fastener portion.

In accordance with another aspect of the disclosure, the first fastener portion can include one of a hook portion or a loop portion, and the second fastener portion can include the other of the hook portion or the loop portion, with the hook portion being configured for releasably fixed attachment to the loop portion.

In accordance with another aspect of the disclosure, the hook portion can be fixed adjacent one of the opposite edges and the loop portion can be fixed adjacent the other of the opposite edges, thereby releasably fixing the opposite edges to one another when the flexible wall is in the closed state.

In accordance with another aspect of the disclosure, the hook portion can be fixed adjacent both of the opposite ends and the loop portion can be fixed adjacent both of the opposite ends.

In accordance with another aspect of the disclosure, the at least one closure member can include a plurality of closure members, with a plurality of the hook portions fixed adjacent one of the opposite edges and a plurality of the loop portions fixed adjacent the other of the opposite edges, and optionally, a single hook portion and a single loop portion can be disposed along the entirety of the opposite edges.

In accordance with another aspect of the disclosure, at least one fixation member can be fixed to an outer surface of the flexible wall, wherein the at least one fixation member is configured to releasably fix the flexible wall against movement relative to an external environment surface, such as, for example, to a drape lying over a patient, over a surgical table, and/or over a Mayo stand.

In accordance with another aspect of the disclosure, the at least one fixation member can include a self-adhesive and a release film releasably covering the self-adhesive.

In accordance with another aspect of the disclosure, the at least one fixation member can include a plurality of fixation members.

In accordance with another aspect of the disclosure, a pocket can be provided to extend along the outer surface of the flexible wall, with the at least one fixation member being fixed to a first side of the flexible wall, and the pocket extending along a second side of the flexible wall, the second side being opposite the first side.

In accordance with another aspect of the disclosure, the pocket can be provided to extend from an open end of the pocket, facing a first one of the opposite ends, to a closed end of the pocket, facing a second one of the opposite ends.

In accordance with another aspect of the disclosure, the open end of the pocket can be spaced from the first one of the opposite ends, thereby allowing easy access and opening of the open end of the pocket, and the closed end can be substantially flush with the second one of the opposite ends.

In accordance with another aspect of the disclosure, an attachment member can be fixed to an inner surface of the pocket adjacent the open end, with the attachment member being configured for attachment to a medical instrument apparatus, such as a holster configured to support a medical instrument in the pocket.

In accordance with another aspect of the disclosure, the attachment member can include a self-adhesive and a release film releasably covering the self-adhesive.

In accordance with another aspect of the disclosure, the pocket can be formed as a monolithic piece of material with the flexible wall, thereby simplifying construction.

In accordance with another aspect of the disclosure, the flexible wall and the pocket can be formed of a transparent polymeric material, thereby allowing visualization of the tube(s) and/or cord(s) disposed within the sleeve.

In accordance with another aspect of the disclosure, the material of the wall and pocket can be provided being between about 3-5 mils thick, and in one non-limiting embodiment, between about 3.5-4.5 mils, thereby making the flexible and pocket highly flexible, yet strong and tear-resistant.

In accordance with another aspect of the disclosure, the pocket has a receptacle bounded by a circumferentially continuous wall, wherein the circumferentially continuous wall can have a first bonded seam extending lengthwise between the opposite ends.

In accordance with another aspect of the disclosure, the receptacle extends from an open end, proximate a first one of the opposite ends of the wall, to a closed end, proximate a second one of the opposite ends of the wall, with the closed end being formed by a second bonded seam extending in inclined relation to the first bonded seam.

In accordance with another aspect of the disclosure, a method of constructing a disposable surgical tube and/or cord containment drape for shielding at least one tube and/or cord against contamination, and optionally, for bundling a plurality of the tube and/or cord with one another, is provided. The method includes, forming a flexible wall having opposite edges extending lengthwise along an axis between opposite ends, with the flexible wall being foldable along the axis from an open state to a closed state to bound an inner cavity by an inner surface of the flexible wall, with the inner cavity being sized for receipt of the surgical tube and/or cord therein. Further, fixing at least one closure member to the flexible wall and configuring the at least one closure member to releasably fix the flexible wall in the closed state.

In accordance with another aspect of the disclosure, the method can further include fixing the at least one closure member to the flexible wall by fixing a first fastener member of the at least one closure member adjacent one of the opposite edges and fixing a second fastener member of the at least one closure member adjacent the other of the opposite edges.

In accordance with another aspect of the disclosure, the method can further include providing the first fastener member being one of a hook or loop portion and the second fastener member being the other of the hook or loop portion, with the hook portion being configured for releasably fixed attachment to the loop portion.

In accordance with another aspect of the disclosure, the method can further include fixing at least one fixation member to an outer surface of the flexible wall, wherein the at least one fixation member is configured to releasably fix the flexible wall against movement relative to an external environment surface.

In accordance with another aspect of the disclosure, the method can further includes providing the at least one fixation member including a self-adhesive and a release film releasably covering the self-adhesive.

In accordance with another aspect of the disclosure, the method can further include forming a pocket extending along the outer surface of the flexible wall, with the at least one fixation member being fixed to a first side of the flexible wall, and the pocket extending along a second side of the flexible wall, said second side being opposite the first side.

In accordance with another aspect of the disclosure, the method can further include forming the pocket extending from an open end, spaced from a first one of the opposite ends, to a closed end, adjacent a second one of the opposite ends.

In accordance with another aspect of the disclosure, the method can further include fixing an attachment member to an inner surface of the pocket adjacent the open end, with the attachment member being configured for attachment to a medical instrument apparatus, such as an instrument holster.

In accordance with another aspect of the disclosure, the method can further include providing the attachment member including a self-adhesive and a release film releasably covering the self-adhesive.

In accordance with another aspect of the disclosure, the method can further include forming the pocket as a monolithic piece of material with the flexible wall by folding a piece of the monolithic piece of material and bonding a free edge of the folded piece to the flexible wall, such as via a welding process.

In accordance with another aspect of the disclosure, a medical drape has at least one disposable surgical tube and/or cord containment drape, referred to hereafter as drape, integrated therewith.

In accordance with another aspect of the disclosure, the medical drape includes a drape layer and at least one surgical tube and/or cord containment drape fixed to the drape layer, with the at least one surgical tube and/or cord containment drape configured to shield at least one tube and/or cord against contamination.

In accordance with another aspect of the disclosure, the at least one surgical tube and/or cord containment drape includes a flexible wall having opposite edges extending lengthwise between opposite ends, with the flexible wall having an open state and a closed state, wherein while the flexible wall is in the open state, a first of the opposite edges is attached to the drape layer, and a second of the opposite edges is configured to be folded over the at least one tube and/or cord into releasably attached relation to the drape layer to bring the flexible wall to the closed state to contain and shield the at least one tube and/or cord in an inner cavity bounded, at least in part, by an inner surface of the flexible wall.

In accordance with another aspect of the disclosure, the inner cavity is bounded along a first side by the inner surface of the flexible wall, and the inner cavity is bounded along an opposite second side by the drape layer.

In accordance with another aspect of the disclosure, a first fastener portion is fixed to the flexible wall adjacent the second of the opposite edges and a second fastener portion fixed to the drape layer, wherein the first fastener portion is configured for releasable attachment to the second fastener portion.

In accordance with another aspect of the disclosure, the first fastener portion is one of a hook portion or a loop portion and the second fastener portion is the other of the hook portion or the loop portion.

In accordance with another aspect of the disclosure, while the flexible wall is in the open state, one of the opposite edges is configured to be folded about the at least one tube and/or cord toward the other of the opposite sides to bring the flexible wall to the closed state to contain and shield the at least one tube and/or cord in an inner cavity bounded entirely by an inner surface of the flexible wall.

In accordance with another aspect of the disclosure, the flexible wall has at least one closure member including a first fastener portion fixed adjacent one of the opposite edges and a second fastener portion fixed adjacent the other of the opposite edges, wherein the first fastener portion is configured for releasably fixed attachment to the second fastener portion to releasably fix the flexible wall in the closed state.

In accordance with another aspect of the disclosure, the at least one surgical tube and/or cord containment drape includes a pocket extending along an outer surface of the flexible wall.

In accordance with another aspect of the disclosure, the at least one surgical tube and/or cord containment drape includes a plurality of surgical tube and/or cord containment drapes attached to one another.

In accordance with another aspect of the disclosure, the medical drape having at least one sleeve is a Mayo stand drape.

In accordance with another aspect of the disclosure, the medical drape having at least one sleeve is a patient drape.

In accordance with another aspect of the disclosure, a disposable surgical tube and/or cord containment drape for shielding at least one tube and/or cord against contamination, and optionally, for bundling a plurality of the tube and/or cord with one another, includes a flexible wall having opposite edges extending lengthwise between opposite ends. A fixation member extends adjacent one of the opposite edges. The fixation member includes a self-adhesive for fixation to a surgical drape. At least one closure member is provided adjacent the other of the opposite edges. The at least one closure member includes a first fastener portion fixed to the flexible wall and a second fastener portion releasably attached to the first fastener portion. The second fastener portion has a self-adhesive facing away from the first fastener portion for fixation to the surgical drape.

In accordance with another aspect of the disclosure, the first fastener portion is one of a hook portion or a loop portion, and the second fastener portion is the other of the hook portion or the loop portion.

In accordance with another aspect of the disclosure, the drape can further include a first release film covering the self-adhesive of the fixation member and a second release film covering the self-adhesive of the second fastener portion, with the first release film being selectively removable to expose the self-adhesive of the fixation member for fixation to the surgical drape, and the second release film being selectively removable to expose the self-adhesive of the second fastener portion for fixation to the surgical drape.

In accordance with another aspect of the disclosure, the drape can further include a pocket extending along an outer surface of the flexible wall.

In accordance with another aspect of the disclosure, the flexible wall of the drape is sterile.

In accordance with another aspect of the disclosure, a method of constructing a disposable surgical tube and/or cord containment drape for shielding at least one tube and/or cord against contamination, and optionally, for bundling a plurality of the tube and/or cord with one another is provided. The method includes, forming a flexible wall having opposite edges extending lengthwise between opposite ends. Further, fixing a fixation member extending adjacent one of the opposite edges, with the fixation member including a self-adhesive for fixation to an external environment surface. Further yet, fixing a first fastener portion to the other of the opposite edges in spaced relation from the fixation member and, releasably attaching a second fastener portion to the first fastener portion, with the second fastener portion being provided having a self-adhesive facing away from the first fastener portion for fixation to the external environment surface.

### BRIEF DESCRIPTION OF THE DRAWINGS

These and other aspects, features and advantages of the invention will become more readily appreciated when considered in connection with the following detailed description of presently preferred embodiments and best mode, appended claims and accompanying drawings, in which:

FIG. 1 is a perspective view of a patient on a patient support surface with a surgical drape disposed over the patient and a plurality of tubes and/or cords laying on the surgical drape, with the plurality of tubes and/or cords shown, by way of example, grouped in discrete bundles, with each bundle shown being protected against contamination by a disposable surgical tube and/or cord containment drape constructed in accordance with an aspect of the disclosure;

FIG. 2 illustrates a schematic plan view of one of the disposable surgical tube and/or cord containment drapes of FIG. 1 prior to completing construction of a pocket of the disposable surgical tube and/or cord containment drape and prior to fixing at least one closure member and at least one attachment member thereto;

FIG. 2A is a view similar to FIG. 2 showing a flap of a monolithic piece of material being folded to form the pocket of the disposable surgical tube and/or cord containment drape;

FIG. 3 illustrates a top schematic plan view of the disposable surgical tube and/or cord containment drape after completing formation of the pocket, with the drape shown in an open state;

FIG. 4 is a view similar to FIG. 3 upon fixing a plurality of closure members and attachment members to the disposable surgical tube and/or cord containment drape;

FIG. 5A illustrates a top schematic perspective view of the disposable surgical tube and/or cord containment drape of FIG. 4, with the drape shown being folded toward a closed state;

FIG. 5B illustrates a top schematic plan view of the disposable surgical tube and/or cord containment drape of FIG. 4, with the drape shown folded to a closed state;

FIG. 5C illustrates a bottom schematic plan view of the disposable surgical tube and/or cord containment drape of FIG. 5B;

FIGS. 6A-6D each illustrate a surgical drape having one or more surgical tube and/or cord containment drapes constructed in accordance further aspects of the disclosure integrated in attached relation therewith;

FIGS. 7A-7D each illustrate a surgical drape having one or more surgical tube and/or cord containment drapes constructed in accordance with further aspects of the disclosure integrated in attached relation therewith;

FIGS. 8A and 8B each illustrate a surgical drape having a plurality of surgical tube and/or cord containment drapes constructed in accordance with further aspects of the disclosure integrated in attached relation therewith;

FIGS. 9A-9C each illustrate a surgical drape having a pair of surgical tube and/or cord containment drapes constructed as a single piece of material in accordance with further aspects of the disclosure integrated in attached relation therewith;

FIG. 10 illustrates a surgical drape having pair of surgical tube and/or cord containment drapes constructed as a single piece of material in accordance with another aspect of the disclosure integrated in attached relation with a surgical drape;

FIG. 11 illustrates a surgical drape having surgical tube and/or cord containment drapes as shown in FIG. 10 and a surgical tube and/or cord containment drape as shown in FIG. 6C integrated in attached relation therewith;

FIG. 12 is a perspective view of a patient on a patient support surface with a surgical stand adjacent the patient support surface, with a surgical drape disposed over the surgical stand, with a plurality of tubes and/or cords laying on the surgical stand, with the plurality of tubes and/or cords shown, by way of example, grouped in discrete bundles, with each bundle shown being protected against contamination by a disposable surgical tube and/or cord containment drape constructed in accordance with an aspect of the disclosure;

FIG. 13 is a perspective view of the surgical stand of FIG. 12, with one of the surgical tube and/or cord containment drapes shown in a deployed, closed state with the bundle protected and contained therein, with the other surgical tube and/or cord containment drapes shown in a non-deployed, open state with the bundle removed therefrom;

FIG. 14 is a perspective view of one of the surgical tube and/or cord containment drapes of FIG. 12 illustrating a pair of release layers partially removed from a pair of self-adhesive strips, wherein the self-adhesive strips are configured for attachment to the surgical drape;

FIG. 14A is a view similar to FIG. 14 illustrating a first fastener portion detached from a second fastener portion of the surgical tube and/or cord containment drape;

FIG. 15 is a perspective view of one of the surgical tube and/or cord containment drapes of FIG. 12, with the surgical tube and/or cord containment drape shown being moved from a non-deployed, open state toward a deployed, closed state to protect and conceal a bundle therein;

FIG. 16 is a view similar to FIG. 13 illustrating a plurality of surgical tube and/or cord containment drapes constructed in accordance with another aspect of the disclosure attached to a surgical drape, with the surgical tube and/or cord containment drapes shown in a deployed, closed state with discrete bundles protected and contained therein;

FIG. 17A is a view similar to FIG. 13 illustrating a pair of surgical tube and/or cord containment drapes constructed as a single piece of material in accordance with another aspect of the disclosure, with the drapes attached to a surgical drape and with a pair of discrete bundles protected and contained by each drape;

FIG. 17B is a view similar to FIG. 17A illustrating the pair of surgical tube and/or cord containment drapes of FIG. 17A shown in a non-deployed, open state;

FIG. 18A illustrates a schematic plan view of a disposable surgical tube and/or cord containment drape constructed in accordance with another aspect of the disclosure, shown prior to completing construction of a pocket of the disposable surgical tube and/or cord containment drape and prior to fixing at least one closure member and at least one attachment member thereto;

FIG. 18B is a view similar to FIG. 18A showing a flap of a monolithic piece of material being folded to form the pocket of the disposable surgical tube and/or cord containment drape; and

FIG. 18C illustrates a top schematic plan view of the disposable surgical tube and/or cord containment drape after completing formation of the pocket and after attaching a closure member and fixation member thereto, with the drape shown in an open state.

### DETAILED DESCRIPTION OF PRESENTLY PREFERRED EMBODIMENTS

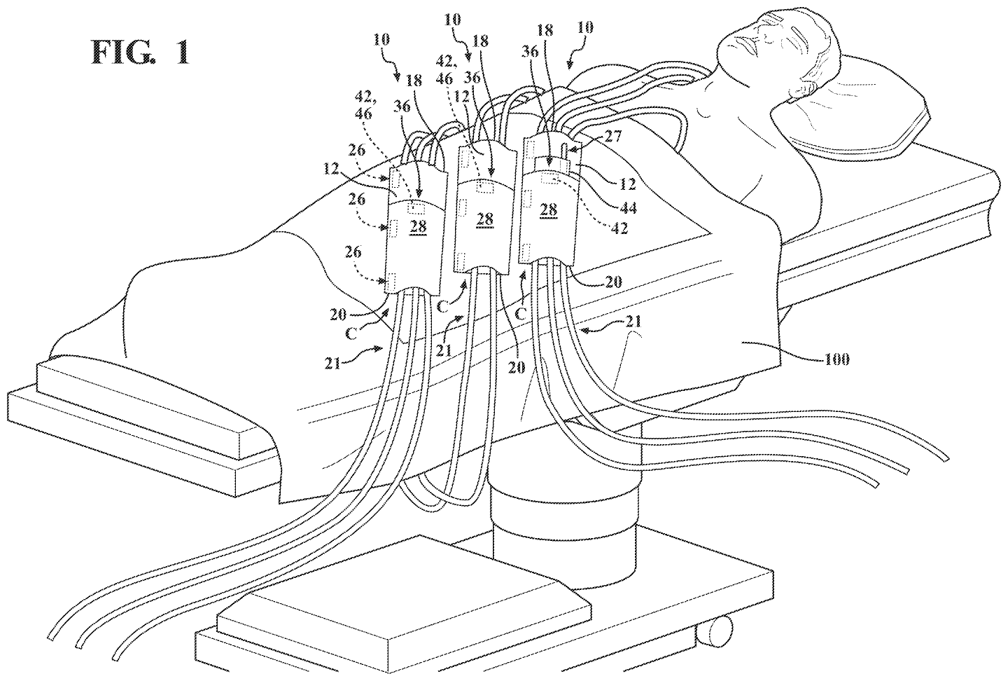

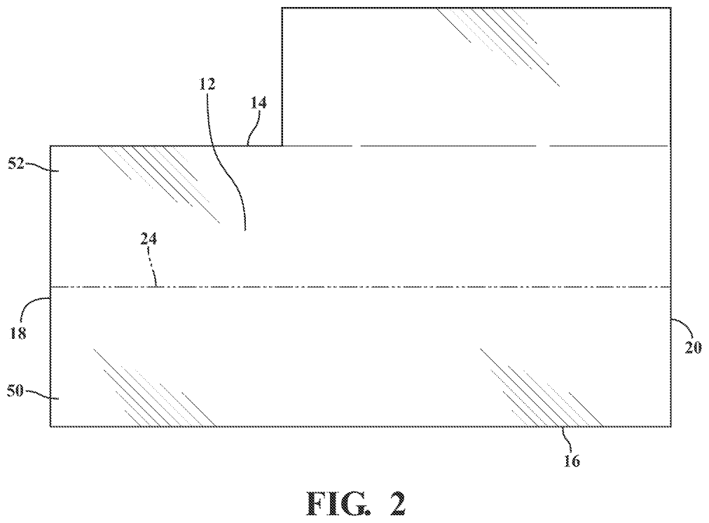

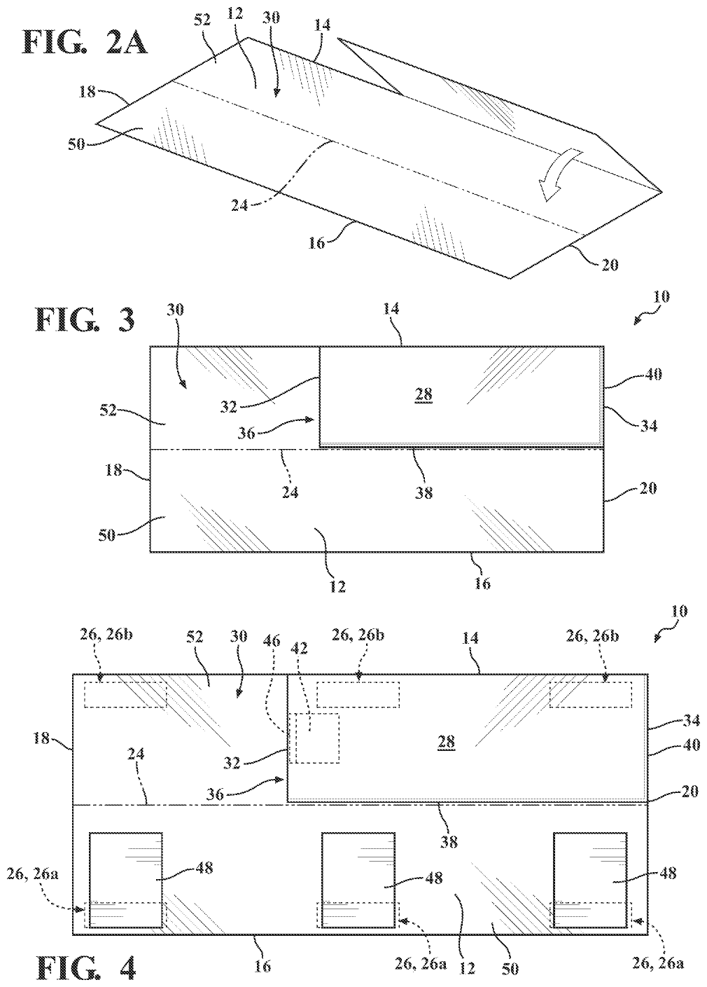

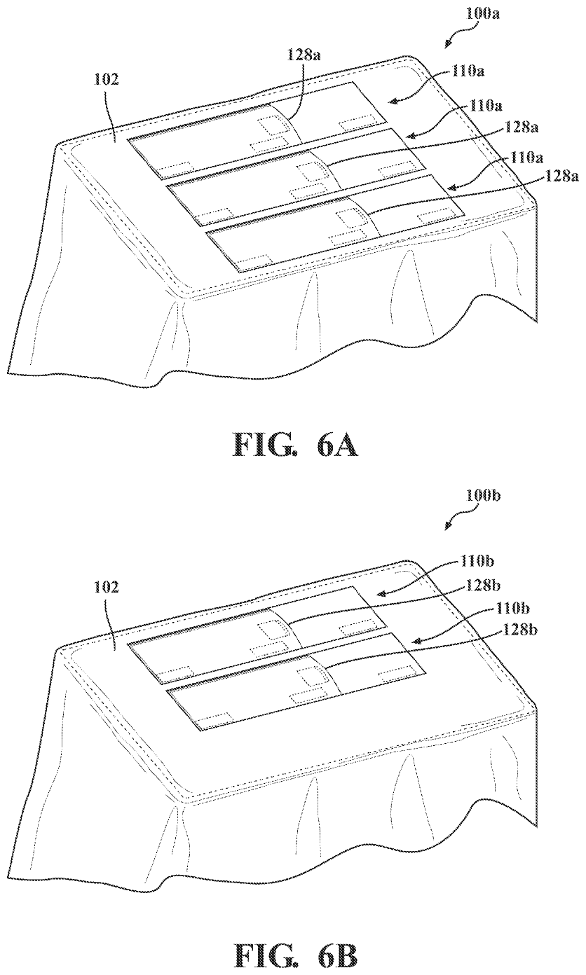

Referring in more detail to the drawings, FIG. 1 illustrates a plurality of disposable sterile, surgical tube and/or cord containment drapes, also referred to as surgical tube and/or cord containment sleeves, or sleeves 10, referred to hereafter in the singular as sleeve, and in the plural as sleeves, constructed in accordance with one presently preferred, non-limiting embodiment of the disclosure. The sleeves 10 includes a sterile, flexible wall 12 having opposite edges 14, 16 (best identified in FIGS. 2-5C) extending lengthwise between opposite ends 18, 20. The flexible wall 12 has a non-deployed, open state, wherein one or more tubes and/or cords 21 can be easily disposed (laid onto, laid across) into the sleeve 10, and a deployed, closed state, wherein the one or more tubes and/or cords 21 are contained (over a length of the sleeve 10 extending between opposite ends 18, 20) and protected in the sleeve 10. While the flexible wall 12 is in the open state, upon laying the desire tube(s) and/or cord(s) 21 along an inner surface 22 (FIG. 5A) of the flexible wall 12, one of the opposite edges 14 is configured to be folded generally along an approximate midsection 24 (best exhibited in FIG. 5A) about the at least one tube and/or cord 21 toward the other of the opposite edges 16 to bring the flexible wall 12 to the closed state to protect, contain and shield the at least one tube and/or cord 21 in an inner cavity C bounded by the inner surface 22 of the flexible wall 12. At least one closure member 26 is fixed to the flexible wall 12, with the at least one closure member 26 being configured to releasably fix the flexible wall 12 in the closed state. The at least one tube and/or cord 21 can be bundled with one another, such as may be desired to group (bundle) similar function tubes/cords 21 with one another, such as suction/shaver tubes/cords, suction, cauterizing tubes/cords, inflow/outflow/light/camera tubes/cords, by way of example and without limitation. By being able to bundle similar function tubes/cords 21 with one another, set-up and tear-down of the surgical arena is greatly simplified, thereby providing great efficiencies in the surgical arena. Further, during a procedure, individual tubes and/or cords can be pulled, as needed, to slide within the cavity C and relative to the sleeve 10, with the sleeve 10 preventing the tubes and/or cords from sliding from a sterile environment, such as is typically defined above a surgical surface of a surgical table and/or above a support surface of a surgical equipment stand, into a non-sterile environment below the sterile environment. Accordingly, sterilization of the tubes and/or cords is maintained by the sleeve 10, thereby avoiding timely tasks associate with replacing contaminated tubes/cords that enter the non-sterile environment.

To facilitate placement of an instrument 27 for ready use by medical personnel, the sleeve 10 can further include a pocket 28 sized to hold the instrument, such as a suction tube, electrosurgical pencil (cauterizing pen), by way of example and without limitation. The pocket 28 extends along an outer surface 30 of the flexible wall 12. The pocket 28 extends from an open end 32 facing a first one of said opposite ends 18, shown as being proximate said end 18, wherein proximate is intended to mean between a midpoint of a length of the wall 12 and the end 18, to a closed end 34 facing a second one of said opposite ends 20, shown as being proximate said end 20, wherein proximate is intended to mean between a midpoint of a length of the wall 12 and the end 20. It is contemplated that the pocket 28 could be open at both ends, and if desired, one of the open ends could be closed, such as via a self-adhesive or otherwise, e.g. hook and loop fastener, but in order to best contain fluids and instruments, it is preferred to have the closed end 34. To facilitate easy opening of the open end 32, and to ensure any portion of the medical instrument extending outwardly from the pocket 28 remains over the wall 12, the open end 32 is spaced from the end 18, e.g. by about ½ inch to several inches, such that a free lip of the open end 32 of the pocket 28 can be readily grasped and opened. Further, to streamline construction, the closed end 34 can be made flush or substantially flush (meaning the end may not be perfectly flush, but within about a millimeter) with the other end 20. The pocket 28 has a suitably sized receptacle 36 bounded by a circumferentially continuous wall, with the circumferentially continuous wall having a first bonded seam 38 extending lengthwise between the opposite ends 32, 34. The closed end 34 of the receptacle 36 can be formed by a second bonded seam 40 along the end 20 of the sleeve 10, such that the second bonded seam extends in inclined relation, shown as being transverse, to the first bonded seam 38. The first and second bonded seams 38, 40 can be formed using any suitable fixation mechanism, and in a preferred embodiment, via adhesive, welding, such as ultrasonic welding, by way of example and without limitation.

To facilitate holding the surgical instrument 27 within the receptacle 36, an attachment member 42 can be fixed to an inner surface of the pocket 28 adjacent the open end 32. The attachment member 42 can be configured for direct attachment to the instrument, or to a medical instrument apparatus, such as a container, also referred to as holster 44. The holster 44 can be provided as desired, such as from molded plastic, by way of example and without limitation. The attachment member 42, in accordance with one presently preferred, non-limiting aspect, includes a self-adhesive and a release film 46 releasably covering the self-adhesive, such that the release film can be readily removed, when desired, thereby exposing the self-adhesive for adhesion to the holster 44. It is contemplated that a hook and loop fastening mechanism could be employed, if desired.

To further streamline construction, thereby reducing costs associated therewith, as shown in FIGS. 2 and 2A prior to completing the formation of the pocket 28, the pocket 28 can be formed as a monolithic piece of material with the flexible wall 12. The material used to form the flexible wall 12 and the pocket 28 can be provided as a sterile, clear (transparent), impervious polymeric material, thus, allowing the contents (tubing, cable, etc.) of the closed sleeve 10 and the contents in the receptacle 36 of the pocket 28 to be easily viewed through the wall 12. The piece(s) of material, whether monolithic or otherwise, can be between about 1-10 mils thick or greater, and in one non-limiting embodiment, between about 2-5 mils thick, by way of example and without limitation, such as from a sterile polyethylene, thereby rendering the wall 12 tough, tear-resistant, and impermeable to fluids.

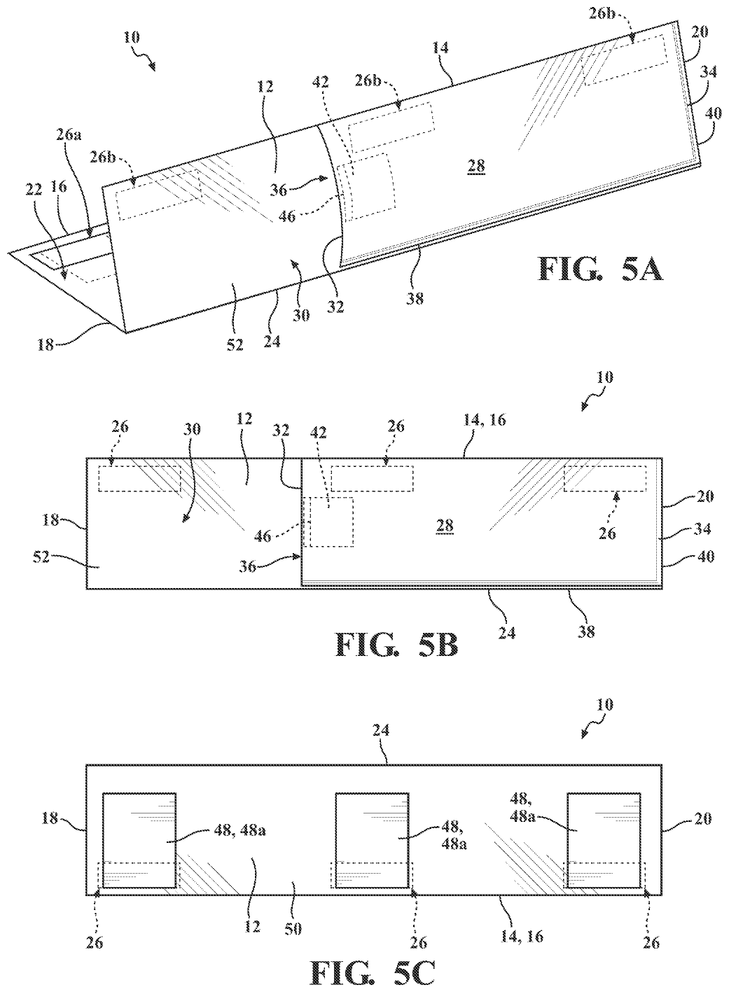

As best shown in FIGS. 4 and 5C, to facilitate holding the sleeve 10 in the desired location, as desired, throughout the medical procedure, at least one fixation member 48 can be fixed to a bottom side, also referred to as first side 50 of the flexible wall 12. The pocket 28 extends along a top side, also referred to as second side 52 of the flexible wall 12, wherein the first side 50 and the second side 52 are opposite one another. The at least one fixation member 48 is configured to releasably fix the flexible wall 12 to and against movement relative to an external environment surface, such as to an external surface of a medical drape, also referred to as surgical drape 100, that is draped over the patient, a surgical stand, also referred to as an instrument stand and commonly known as a Mayo stand, surgical table, or the like. In a non-limiting embodiment, the at least one fixation member 48 is provided as a plurality of fixation members, with each fixation member 48 including a self-adhesive and a release film 48a releasably covering the self-adhesive, such as discussed above for the attachment member 42. Accordingly, when desired, one or more of the release films can be simply pealed from the underlying self-adhesive, whereupon the self-adhesive can be bonded to the desired surface.

As best shown in FIG. 4, the at least one closure member 26 used to releasably maintain the flexible wall 12 in its closed state can be provided including a first fastener portion, such as a hook portion 26a, and a second fastener portion, such as a loop portion 26b, with the (first portion) hook portion 26a being configured for releasably fixed attachment to the (second portion) loop portion 26b. The hook portion 26a is fixed adjacent one of the opposite edges, shown as edge 16, and the loop portion 26b is fixed adjacent the other of the opposite edges, shown as edge 14, by way of example and without limitation. In the exemplary, non-limiting illustrated embodiment, one hook portion 26a is fixed adjacent both of the opposite ends 18, 20, and one loop portion 26b is fixed adjacent both of the opposite ends 18, 20. A further hook portion 26a is provided intermediate (between) the hook portions 26a adjacent the ends 18, 20, and a further loop portion 26b is provided intermediate (between) the loop portions 26b adjacent the ends 18, 20. Accordingly, a plurality of the hook portions 26a are fixed along adjacent one of the opposite edges 16 and a plurality of the loop portions 16b are fixed along and adjacent the other of the opposite edges 14, with the respective hook and loop portions 26a, 26b being located for releasable attachment to one another. The first and second fastener portions 26a, 26b are configured for intentional, releasable attachment to one another, such that the first and second fastener portions 26a, 26b remain attached to one another unless intentionally separated from one another. Separation of the first and second fastener portions 26a, 26b from one another may be desired, such as when disposing the tubes/cords 21 into protected relation with the drape 10, or when desired to remove or service the tubes/cords 21. Accordingly, when desired, quick and easy access to the readily viewable tubes/cords 21 is provided via intentional separation of the first and second fastener portions 26a, 26b from one another.

In accordance with another aspect of the disclosure, a method of constructing a disposable surgical tube and/or cord containment sleeve 10 or shielding at least one tube and/or cord 21 against contamination, and optionally, for bundling a plurality of the tube and/or cord 21 with one another, is provided. The method includes, forming a flexible wall 12 having opposite edges 14, 16 extending lengthwise along an axis 24 between opposite ends 18, 20, with the flexible wall 12 being foldable along the axis 24 from an open state to a closed state to bound an inner cavity C by an inner surface 22 of the flexible wall 12, with the inner cavity C being sized for receipt of the surgical tube and/or cord 21 therein. Further, fixing at least one closure member 26 to the flexible wall 21 and configuring the at least one closure member 26 to releasably fix the flexible wall 12 the closed state.

In accordance with another aspect of the disclosure, the method can further include fixing the at least one closure member 26 to the flexible wall 12 by fixing a first fastener portion, also referred to as first fastener member 26a, of the at least one closure member 26 adjacent one of the opposite edges 16 and fixing a second fastener portion, also referred to as second fastener member 26b, of the at least one closure member 26 adjacent the other of the opposite edges 14.

In accordance with another aspect of the disclosure, the method can further include providing the first fastener member being a hook portion 26a and the second fastener member being a loop portion 26b, with the hook portion 26a being configured for releasably fixed attachment to the loop portion 26b. It is contemplated herein that the first and second fastener portions 26a, 26b could be snaps or other male and female type fastener members.

In accordance with another aspect of the disclosure, the method can further include fixing at least one fixation member 48 to an outer surface 30 of the flexible wall 12, wherein the at least one fixation member 48 is configured to releasably fix the flexible wall 12 against movement relative to an external environment surface, such as a drape over a patient, a Mayo stand, a Mayo stand drape, and/or a surgical table, by way of example and without limitation.

In accordance with another aspect of the disclosure, the method can further includes providing the at least one fixation member 48 including a self-adhesive and a release film releasably covering the self-adhesive.

In accordance with another aspect of the disclosure, the method can further include forming a pocket 28 extending along the outer surface 30 of the flexible wall 12, with the at least one fixation member 48 being fixed to a first side 50 of the flexible wall 12, and the pocket 28 extending along a second side 52 of the flexible wall 12, with the second side 52 being opposite the first side 50.

In accordance with another aspect of the disclosure, the method can further include forming the pocket 28 extending from an open end 32 of the pocket 28, spaced from a first one of the opposite ends 18 of the sleeve 10, to a closed end 34, adjacent a second one of the opposite ends 20 of the sleeve 10.

In accordance with another aspect of the disclosure, the method can further include fixing an attachment member 42 to an inner surface of the pocket 28 adjacent the open end 32, with the attachment member 42 being configured for attachment to a medical device container, such as an instrument holster 44.

In accordance with another aspect of the disclosure, the method can further include providing the attachment member 42 including a self-adhesive and a release film releasably covering the self-adhesive.

In accordance with another aspect of the disclosure, the method can further include forming the pocket 28 as a monolithic piece of material with the flexible wall 12 by folding a piece of the monolithic piece of material and bonding a free edge of the folded piece to the flexible wall 12, such as via any desired welding, heat-sealing process, by way of example and without limitation.

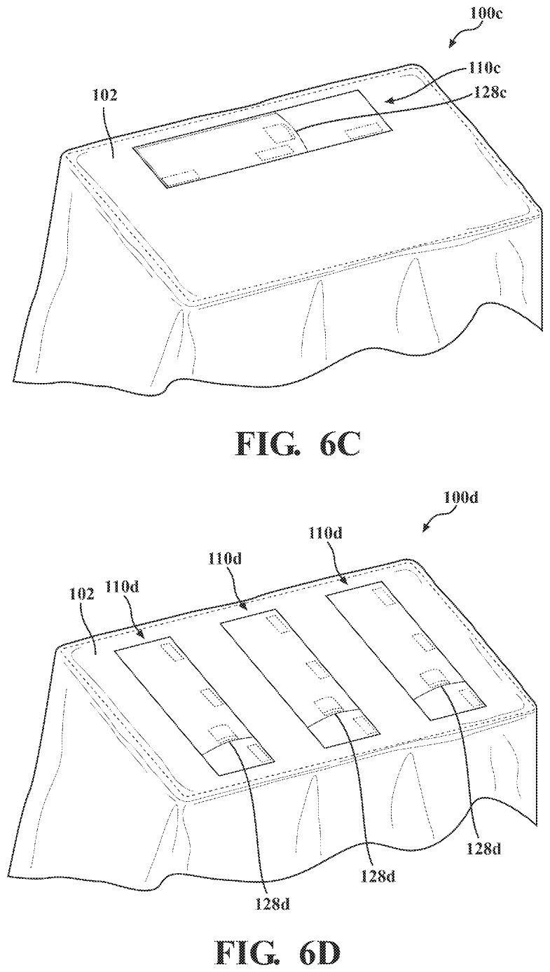

In accordance with another aspect of the disclosure, FIGS. 6A-6D illustrate sterile surgical drapes, such as discussed above, and shown as Mayo stand drapes, by way of example and without limitation, and identified by respective numerals 100a, 100b, 100c, 100d, configured to drape an instrument support surface of a Mayo stand. The drape 100a in FIG. 6A is shown as having a plurality of sterile drapes, also referred to as sleeves 110a, integrated therewith, wherein the sleeves 110a are constructed as discussed above for sleeve 10. As such, it is to be recognized that the sleeves 110a can be fixed to a drape layer 102 of drape 100a via one or more fixation member 48 (see FIGS. 4 and 5C), or otherwise, as desired. The drape layer 102 can be sized as desired, depending on the application, whether for a surgical table, bed, Mayo stand, or otherwise. The drape layer 102 can be fabricated on any suitable, well known surgical drape material, including non-woven, spun-bond material, polymeric material, and in a non-limiting example, from a hydro-entangled polypropylene face fabric laminated to a microporous breathable film which acts as a barrier to most types of micro-organisms and viruses, by way of example and without limitation. The drape layer 102 can be opaque, while the material of the sleeves 110a is clear or transparent, as discussed above, thereby allowing visualization of the contents of the sleeves 110a to ensure no problems exist with the contents of the sleeve 110a. It is further contemplated that the drape layer 102 could be fabricated of the same material as the sleeve 110a in integrated fashion. As can be seen in FIGS. 6A-6C, a plurality of sleeves 110a (FIG. 6A), 110b (FIG. 6B) or one sleeve 110c (FIG. 6C) can be provided on the drapes 100a, 100b, 100c, respectively, wherein the illustrated drapes 100a, 100b, 100c are configured to be disposed over a Mayo stand. In FIGS. 6A-6C, the sleeves 110a, 110b, 110c are shown having pockets 128a, 128b, 128c, as discussed above for pocket 28, and extending lengthwise along a length direction of the drapes 100a, 100b, 100c, while in FIG. 6D, the sleeve 110d is shown having a pocket 128d, with the sleeve 110d extending widthwise along a width direction of the drape 100d and width direction of the Mayo stand.

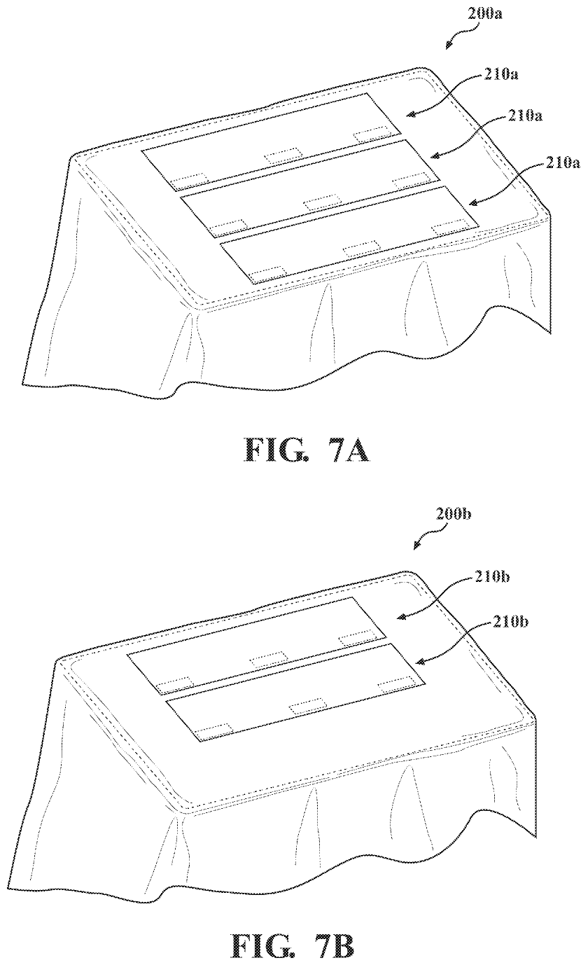

In FIGS. 7A-7D, surgical drapes, such as Mayo stand drapes, identified by respective numerals 200a, 200b, 200c, 200d, configured to drape a surface of a Mayo stand in accordance with another aspect of the disclosure are illustrated. The drapes 200a, 200b, 200c, 200d are similar to the drapes 100a, 100b, 100c, 100d; however, the drapes, also referred to as sleeves 210a, 210b, 210c, 210d, do not have pockets 128a, 128b, 128c, 128d. Otherwise, the drapes 200a, 200b, 200c, 200d are constructed the same as discussed above for drapes 100a, 100b, 100c, 100d.

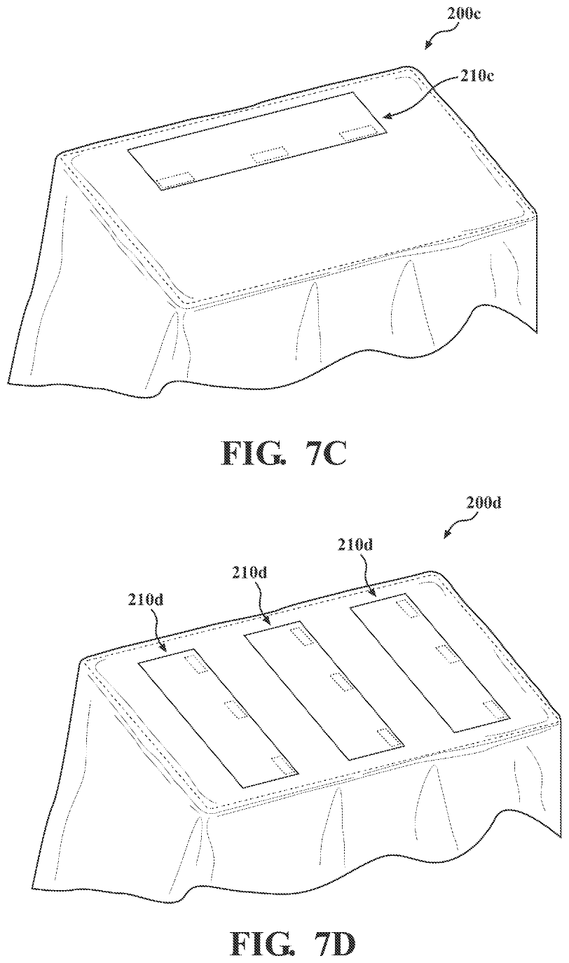

In FIGS. 8A and 8B, surgical drapes, such as Mayo stand drapes, identified by respective numerals 300a, 300b, configured to drape a surface of a Mayo stand in accordance with another aspect of the disclosure are illustrated. The drapes 300a, 300b include one or more drapes, also referred to as sleeves, having pockets, similar to sleeves 110a, 110b, 110c, 110d, and one or more sleeves not having pockets similar to the sleeves 210a, 210b, 210c, 210d. In particular, the drape 300a of FIG. 8A includes one sleeve 310a having a pocket 328a and one sleeve 310a′ not having a pocket, while the drape 300b of FIG. 8B includes two sleeves 310b′ not having a pocket and one sleeve 310b having a pocket 328b. Otherwise, the drapes 300a, 300b are constructed the same as discussed above for drapes 100a-100d and 200a-200d.

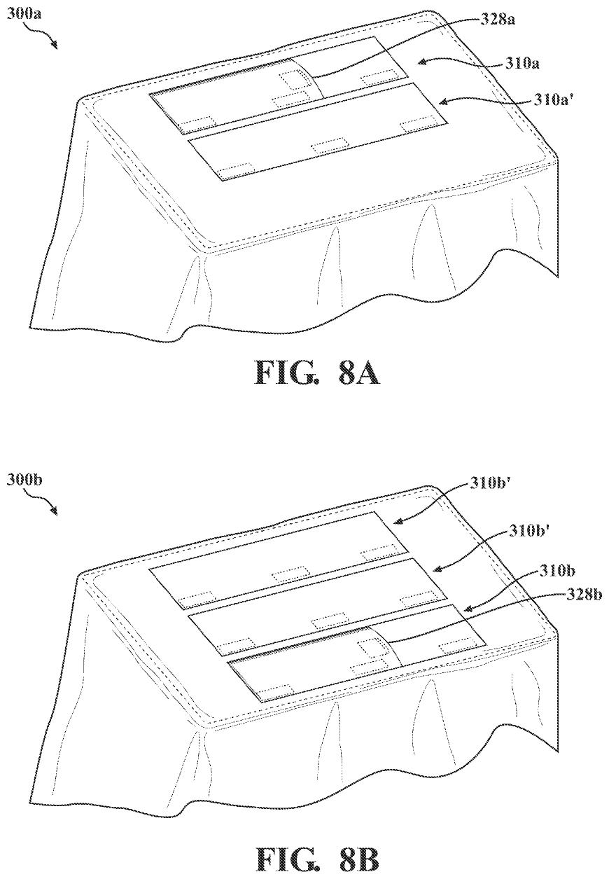

In FIGS. 9A-9C, surgical drapes, such as Mayo stand drapes, identified by respective numerals 400a, 400b, 400c, configured to drape a surface of a Mayo stand in accordance with another aspect of the disclosure are illustrated. The drape 400a of FIG. 9A includes two sleeves 410a in side-by-side, connected relation with one another. The sleeves 410a do not include pockets, and can be connected via a heat-seal, also referred to as heat-stake, in the polymeric material extending along a mid-section between the two sleeves 410a. The drape 400b of FIG. 9B is similar to drape 400a, including two sleeves 410b, 410b′ in side-by-side, connected relation with one another via a heat-seal in the polymeric material extending along a mid-section between the two sleeves 410b. However, rather than neither sleeve having a pocket, one sleeve 410b has a pocket 428b, while the other sleeve 410b′ does not have a pocket. Otherwise, the drape 400b is the same as drape 400a. The drape 400c of FIG. 9C is similar to drape 400b, including two sleeves 410c, 410c in side-by-side, connected relation with one another via a heat-seal in the polymeric material extending along a mid-section between the two sleeves 410b. However, rather than only one sleeve having a pocket, both sleeves 410c have a pocket 428c. Otherwise, the drape 400c is the same as drape 400b.

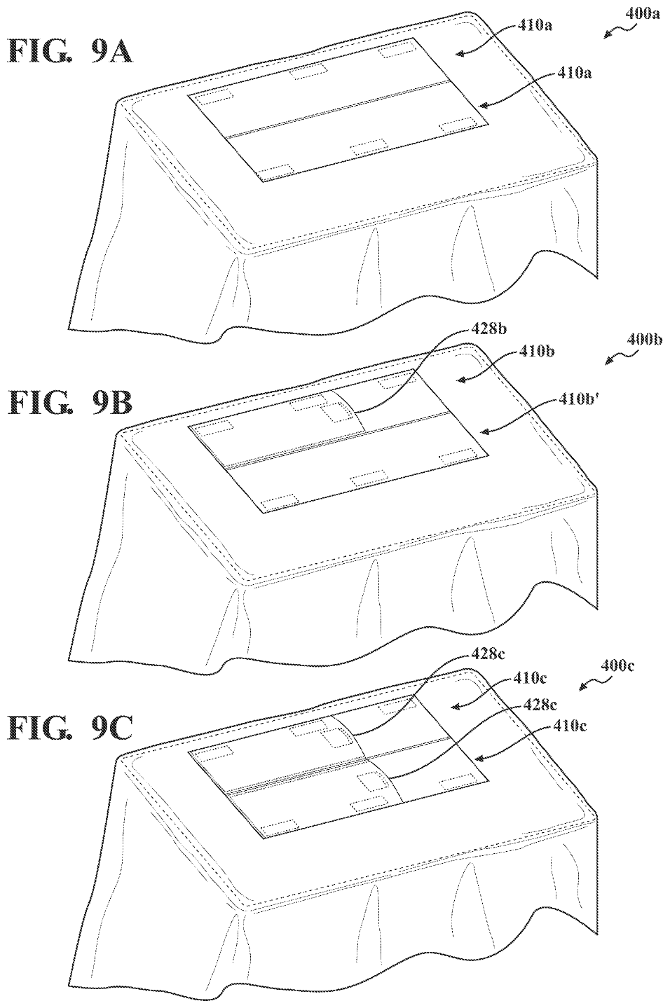

In FIG. 10, a surgical drape, such as a Mayo stand drape, identified by numeral 500 configured to drape a surface of a Mayo stand in accordance with another aspect of the disclosure is illustrated. The drape 500 is similar to drape 400, including two sleeves 510 in side-by-side, connected relation with one another, wherein the sleeves 510 can be fixed to a drape layer 502 of the drape 500 via self-adhesive strip(s) as discussed above for fixation members 48. The sleeves 510 do not include pockets, but rather than being connected via a heat-seal, as with drape 400, the sleeves 510 of drape 500 are connected via a common, single piece bottom side, also referred to as first side 550, with the top side, also referred to as second side, having a pair of folded second sides 552 to form the pair of receptacles 536. The pair of folded second sides 552 are shown as bi-folds, with each side 536 folding inwardly toward one another to bring closure members 526, such as discussed above for closure members 26, into releasably attached relation with one another to form the separate receptacles 536.

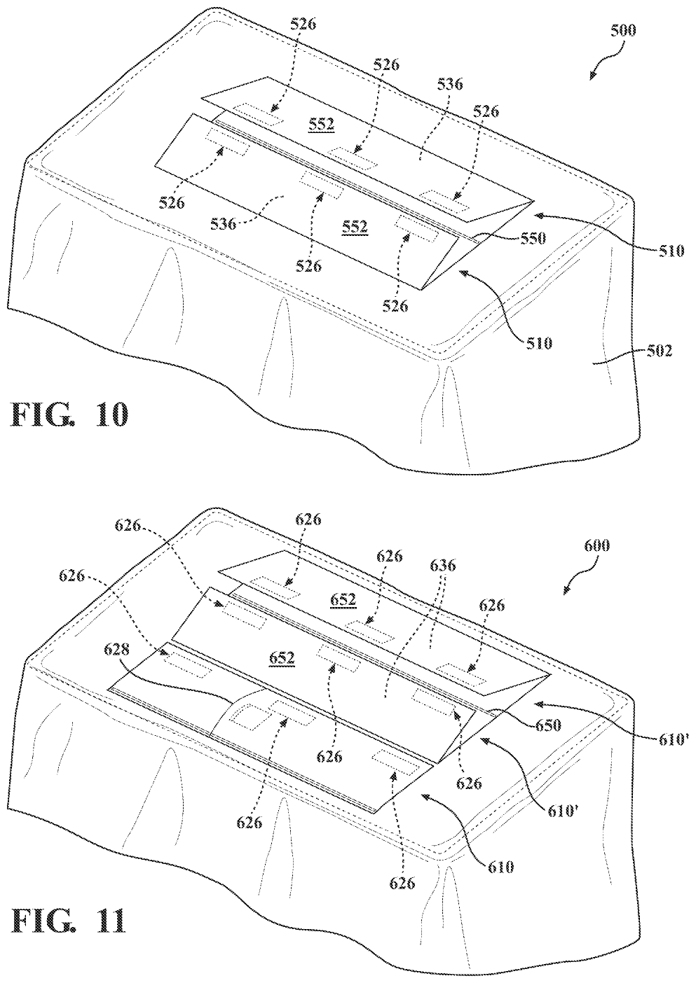

In FIG. 11, a surgical drape, such as a Mayo stand drape, identified by numeral 600 configured to drape a surface of a Mayo stand in accordance with another aspect of the disclosure is illustrated. The drape 600 is similar to drape 500, including two sleeves 610′ in side-by-side, connected relation with one another. The sleeves 610′ do not include pockets and are connected via a common, single piece bottom side, also referred to as first side 650, with the top side, also referred to as second side, having a pair of second sides 652 foldable toward one another to bring closure members 626 into releasably attached relation with one another to form the pair of receptacles 636. Drape 600 further includes a sleeve 610 with a pocket 628 extending adjacent the sleeves 610′. Otherwise, drape 600 is the same as drape 500.

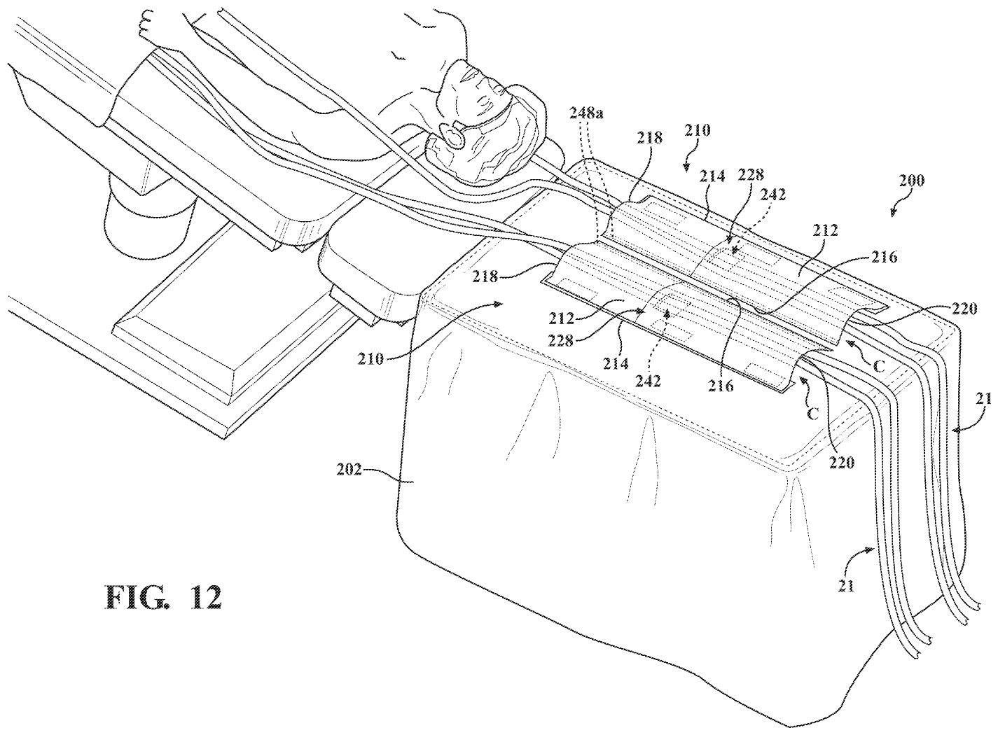

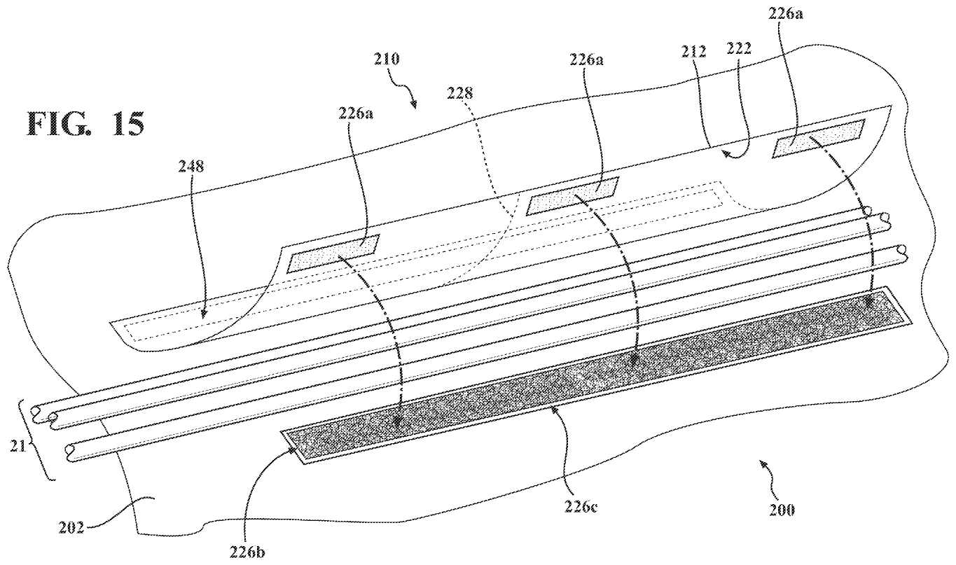

In accordance with another aspect of the disclosure, FIGS. 12-15 illustrate a sterile surgical drape, such as discussed above, and shown as a Mayo stand drape 200, by way of example and without limitation, configured to drape an instrument support surface of a Mayo stand. The surgical drape 200 has a drape layer 202 with at least one, and shown a pair of surgical tube and/or cord containment drapes, referred to hereafter as drapes 210, attached thereto. The drapes 210 can be integrated with the drape layer 202, thereby forming a surgical drape 200 as an assembly ready for use, or the drapes 210, as detailed hereafter, can be provided separately and subsequently fixed to the drape layer 202 for use when desired. Accordingly, the drape layer 202 and the drapes 210 can be stocked separately from one another for use together or separately from one another, when desired.

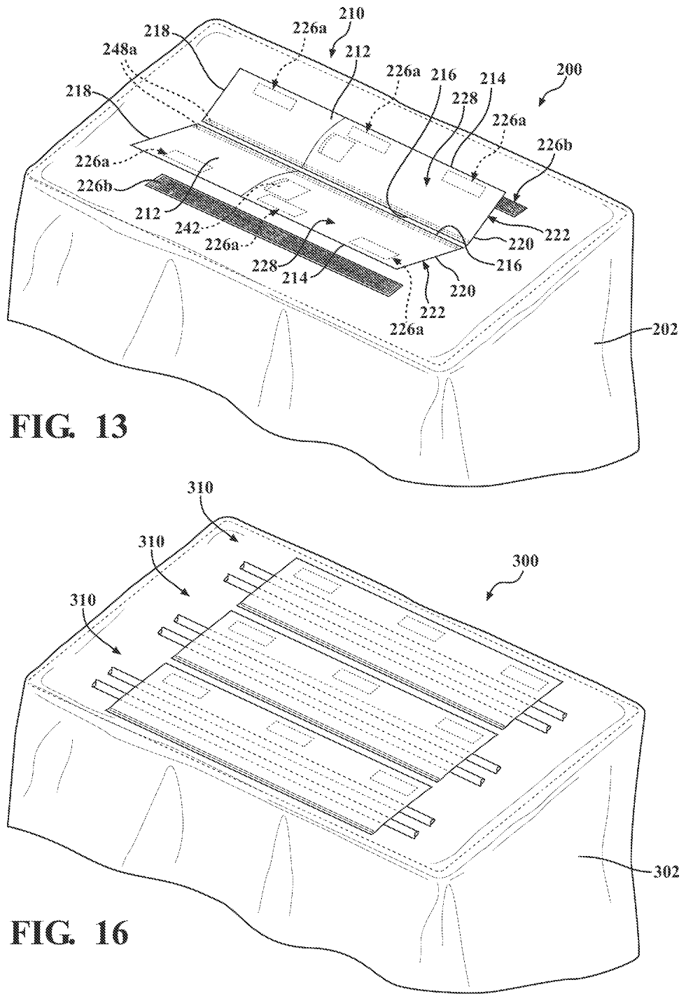

The drapes 210, and referred to hereafter in the singular as drape 210 for description purposes only, includes a flexible wall 212, constructed of the same material discussed above for flexible wall 12, having opposite edges 214, 216 extending lengthwise between opposite ends 218, 220, with the flexible wall 212 having an open state (FIG. 13) and a closed state (FIG. 12). While the flexible wall 212 is in the open state, a first of the opposite edges 216 is shown attached to the drape layer 202, and a second of the opposite edges 214 detached from the flexible wall 212 and is configured to be disposed, referred to as folded, over at least one tube and/or cord 21 into releasably attached relation to the drape layer 202 to bring the flexible wall 212 to the closed state to contain and shield the at least one tube and/or cord 21 in an inner cavity C bounded, at least in part, by an inner surface 222 of the flexible wall 212. In the illustrated embodiment, the cavity C is bounded along a first side by the inner surface 222 of the flexible wall 212, and the inner cavity C is bounded along a second side by the drape layer 202. Accordingly, the at least one tube and/or cord 21 is releasably captured and contained in sandwiched relation between the flexible wall 212 and the drape layer 202.

The drape 210 includes at least one fastener member, referred to as fixation member 248, extending adjacent the edge 216 and at least one closure member, and shown as a plurality of closure members 226, by way of example and without limitation, adjacent the other edge 214. The fixation member 248 can be provided as a self-adhesive layer 248a configured for direct fixation to the drape layer 202. Upon being adhered directly to the drape layer 202, the drape 210 is intended to remain fixed to the drape layer 202, as it is typically required by operating room protocol to avoid removing an article from direct fixation from a drape layer. Prior to fixing the self-adhesive layer 248a to the drape layer 202, a release film 248b can be provided over the self-adhesive layer 248a to protect the self-adhesive layer 248a against contamination, and then, when desired to fix the self-adhesive layer 248a to the drape layer 202, the release film 248b can be removed.

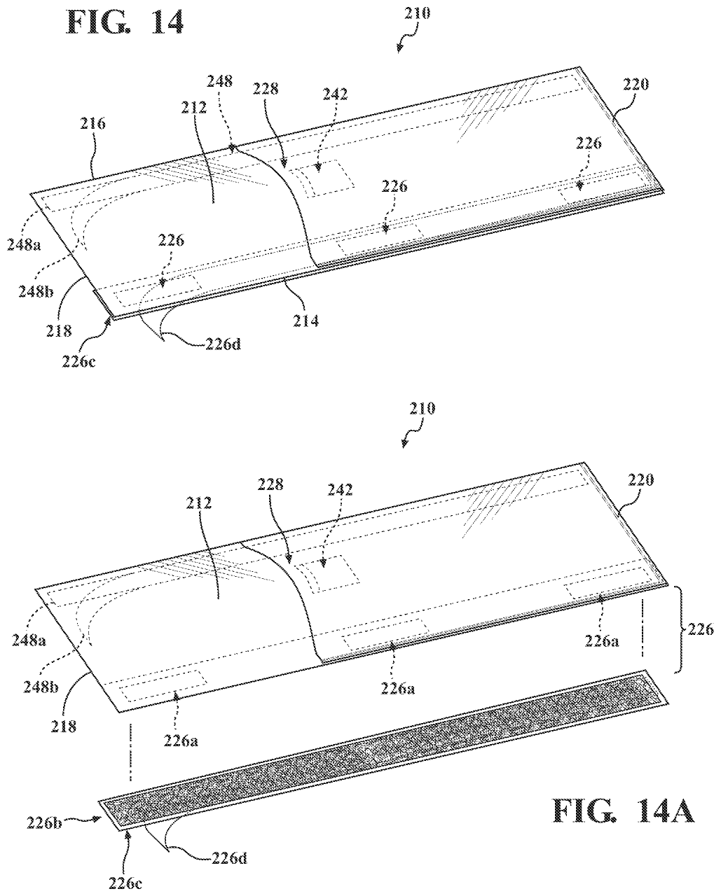

The closure members 226 are illustrated having a first fastener portion 226a fixed to the flexible wall 212 adjacent the opposite edge 214 and a second fastener portion 226b fixed to the drape layer 202. The first fastener portion 226a is configured for releasable attachment to the second fastener portion 226b, thereby allowing the edge 214 to be releasably fixed to the drape layer 202, and subsequently detached from the drape layer 202, as may be desired to service or otherwise tend to the at least one tube and/or cord 21 during a medical procedure. It is to be recognized that separation of the first and second fastener portions 226a, 226b from one another is permitted due to the second fastener portion 226b remaining fixed to the drape layer 202, while the first fastener portion 226 is detached from the second fastener portion 226b. The first fastener portion 226a can be provided as one of a hook portion or a loop portion of a hook and loop type fastener, and the second fastener portion 226b can be provided as the other of the hook portion or the loop portion of the hook and loop type fastener. The second fastener portion 226b can be provided having a self-adhesive layer 226c (FIGS. 14 and 14A) along an opposite side from the hook or loop portion, facing away from the first fastener portion 226a and toward the drape layer 202, with the self-adhesive layer 226c being configured for direct, permanent fixation to the drape layer 202. Upon being adhered directly to the drape layer 202, the drape 210 is intended to remain fixed to the drape layer 202, as it is typically required by operating room protocol to avoid removing an article from direct fixation from a drape layer, as this can compromise the material of the drape layer 202, such as by tearing or weakening the protective function of the drape layer 202. Prior to fixing the self-adhesive layer 226c to the drape layer 202, a release film 226d can be provided over the self-adhesive layer 226c to protect the self-adhesive layer 226c against contamination, and then, when desired to fix the self-adhesive layer 226c to the drape layer 202, the release film 226d can be removed. In the embodiment illustrated, the second fastener portion 226b is shown as a continuous, single strip of material, though it is to be recognized that individual, separate pieces could be used corresponding in number with the first fastener portions 226a. It is to be further understood that the first fastener portions 226, although shown as a plurality of individual, separate pieces of material, could be provided as a single strip of material, if desired. Further yet, it is to be recognized that the fixation member 248 could be provided in similar fashion as described for the closure members 226, having first and second fastener portions, though this would likely add cost and complexity the drape 212 and drape 202 therewith.

The drape 210 can include a pocket 228 extending along an outer surface 230 of the flexible wall 212. The pocket 228 can be formed as discussed above for pocket 28, or otherwise, as desired. The pocket 228 can also include an attachment member 242, as discussed above for attachment member 42.

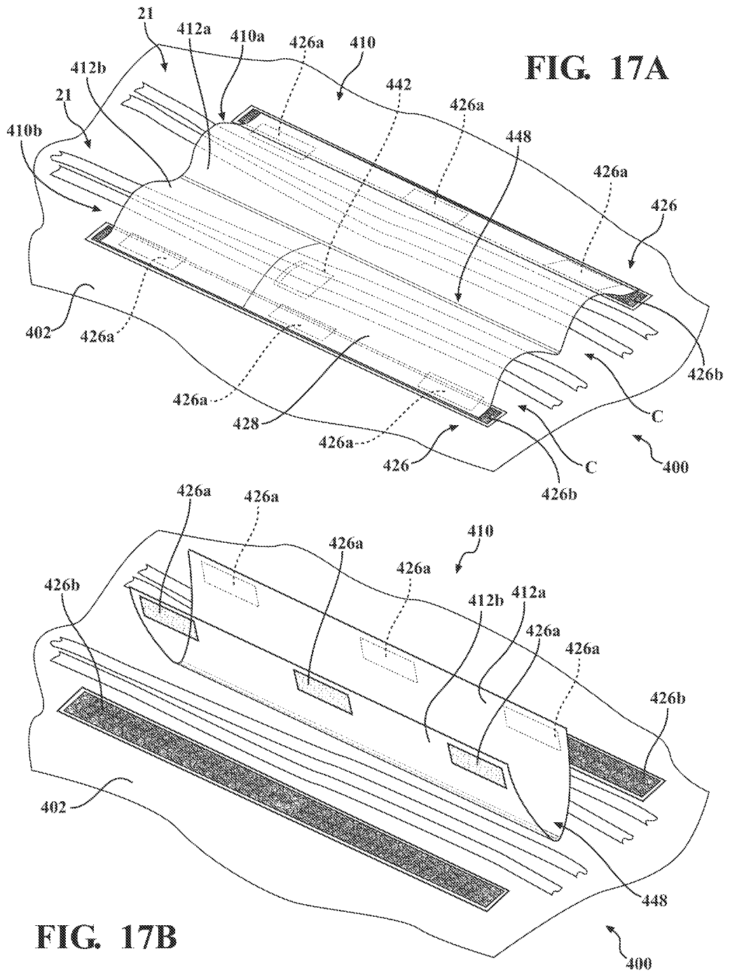

In accordance with another aspect of the disclosure, FIG. 16 illustrates a surgical drape, such as discussed above, and shown as a Mayo stand drape 300, by way of example and without limitation, configured to drape an instrument support surface of a Mayo stand. The surgical drape 300 has a drape layer 302 with at least one, and shown a pair of surgical tube and/or cord containment drapes, referred to hereafter as drapes 310, attached thereto. The drapes 310 are similar to the drapes 210, however, the drapes 310 do not include pockets, as discussed above for pockets 28, 228. Otherwise, the drapes 310 are the same as drapes 210, and thus, no further discussion is believed necessary to fully understand the drapes 310 and functionality thereof.

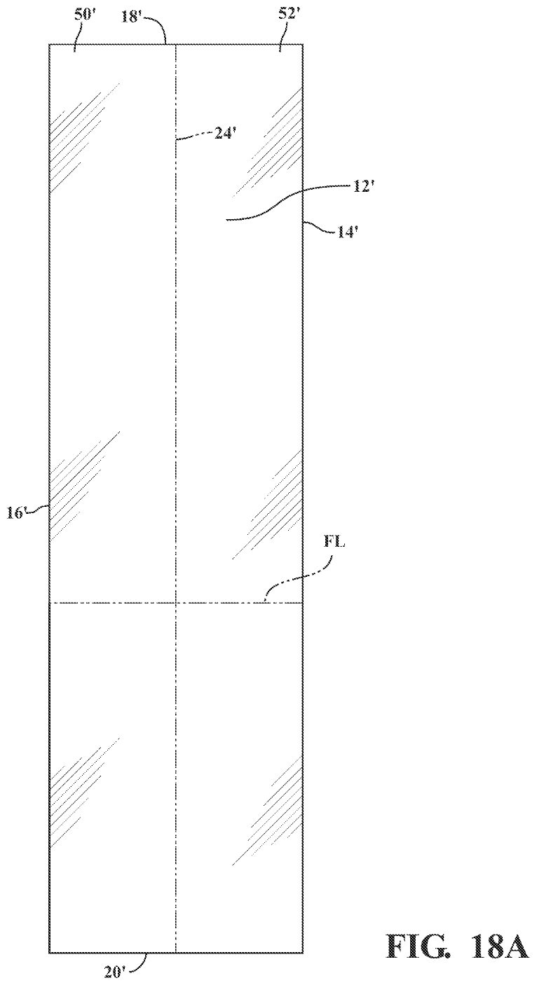

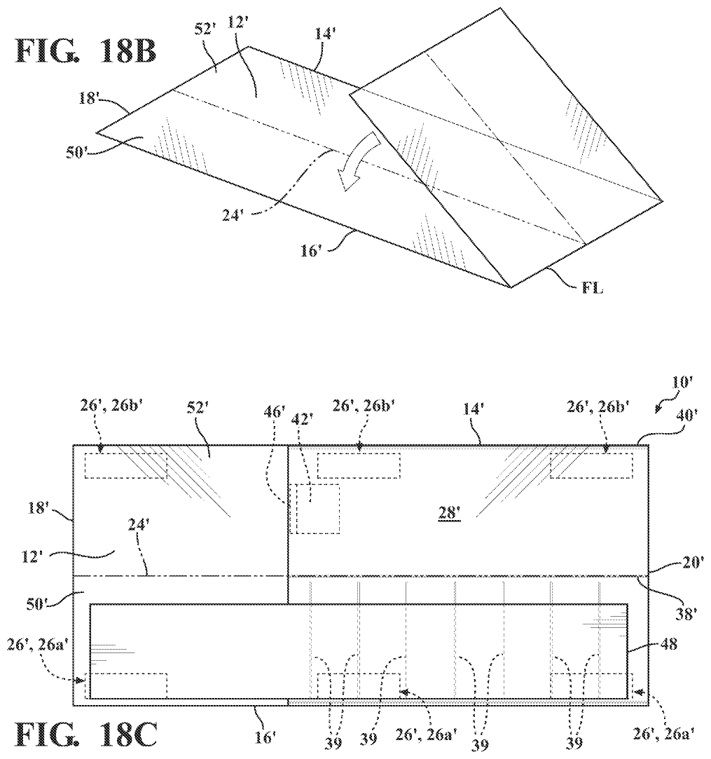

In accordance with another aspect of the disclosure, FIGS. 17A and 17B illustrate a surgical drape, such as discussed above, and shown as a Mayo stand drape 400, by way of example and without limitation, configured to drape an instrument support surface of a Mayo stand. The surgical drape 400 has a drape layer 402 with a surgical tube and/or cord containment drape, referred to hereafter as drape 410, attached thereto. The drape 410 is formed of a single piece of material forming a wall 412, with the wall 412 defining a pair of drapes 410a, 410b in side-by-side relation, with one of the drapes 410b having a pocket 428. The pocket 428 can be formed via any suitable method of construction, such as discussed above for pocket 28, or as discussed below for FIGS. 18A-18C. The wall 412 has a central fixation member 448, such as discussed above for fixation member 48, with each drape 410a, 410b extending laterally away from the fixation member 448. Accordingly, a pair of drapes 410a, 410b for containing and shielding different bundles of tubes and/or cords 21 in separate, side-by-side inner cavities C is provided.

In application, the fixation member 448 can be fixed directly to the drape layer 402, whereupon the separate drapes 410a, 410b are in an open, non-deployed state (FIG. 17B). The separate bundles of tubes and/or cords 21 can be disposed beneath the desire wall 412a, 412b, and then the walls 412a, 412b can be moved to their closed, deployed states. To releasably maintain the walls 412a, 412b in their closed, deployed states, one or more closure members 426 can be used, with closure members 426 being provided as discussed above for closure members 226, by way of example and without limitation. Accordingly, closure members 426 can include first fastener portions 426a fixed to the flexible walls 412a, 412b and second fastener portions 426b fixed to the drape layer 402. The first fastener portions 426a are configured for releasable attachment to and from the second fastener portions 426b, as discussed above for fastener portions 226a, 226b, thereby allowing a personnel in the medical theater to tend to the tubes and/or cords 21 during a medical procedure, while the second fastener portions 426b remain fixed to the drape layer 402.

In accordance with another aspect of the disclosure, a method of constructing a disposable surgical tube and/or cord containment drape 210 for shielding at least one tube and/or cord 21 against contamination, and optionally, for bundling a plurality of the tube and/or cord 21 with one another is provided. The method includes, forming a flexible wall 212 having opposite edges 214, 216 extending lengthwise between opposite ends 218, 220; fixing a fixation member 248 extending adjacent one of the opposite edges 216, with the fixation member 248 including a self-adhesive 248a for fixation to an external environment surface 202; fixing a first fastener portion 226a to the other of the opposite edges 214 in spaced relation from the fixation member 248; releasably attaching a second fastener portion 226b to the first fastener portion 226a, with the second fastener portion 226b being provided having a self-adhesive 226c facing away from the first fastener portion 226a for fixation to the external environment surface 202.

FIGS. 18A-18C illustrate a drape 10′ constructed in accordance with yet another non-limiting aspect of the disclosure. To further streamline construction, thereby reducing costs associated therewith, not only is a pocket 28′ formed as a monolithic piece of material with the flexible wall 12′, but sheet of material used to form the drape 10′, including the pocket 28′, starts with a rectangular piece of material having opposite edges 14′, 16′ extending between opposite ends 18′, 20′, rather than having a flap extending therefrom, as discussed above with reference to FIG. 2. As shown in FIG. 18B, a portion of the rectangular sheet is folded along a fold line FL to form the drape 10′ having the desired length, as well as the pocket 28′ having the desired length. Upon folding the sheet, formation of the pocket 28′ can be completed via a suitable fixation process, such as via a heat-seal process to form bonded seams along opposite, lengthwise extending edges 38′, 40′. Further, one or more (plurality) attachments can be made to fix the remaining portion of the folded sheet, adjacent the pocket 28′, to the underlying portion of the sheet, and in the embodiment illustrated a plurality of bonded seams 39 are formed to fix the overlying sheet portions to one another. Further, one or more closure members 26′, such as discussed above for closure members 26, are provided, thereby having first and second fastener members 26a′, 26b′ arranged for releasable attachment to one another adjacent the opposite edges 14′, 16′. To facilitate holding the drape 10′ in the desired location, at least one fixation member 48′ can be fixed to a bottom side, also referred to as first side 50′ of the flexible wall 12′. The pocket 28′ extends along a top side, also referred to as second side 52′ of the flexible wall 12′, wherein the first side 50′ and the second side 52′ are opposite one another. The fixation member 48′ is shown as a single fixation member, by way of example and without limitation, with the fixation member 48′ being configured to releasably fix the flexible wall 12′ to and against movement relative to an external environment surface, as discussed above. In use, the flexible wall 12′ is folded about an approximate midsection 24′ to enclose the tubes/cords desired to protect, as discussed above for FIG. 5A. An attachment member 42′ and release film 46′ therefor can also be provided, as discussed above.

Obviously, many modifications and variations of the present invention are possible in light of the above teachings. It is contemplated that all features of all claims and of all embodiments can be combined with each other, so long as such combinations would not contradict one another. It is, therefore, to be understood that within the scope of the appended claims, the invention may be practiced otherwise than as specifically described.

What is claimed is:

1. A disposable surgical tube and/or cord containment drape for shielding at least one tube and/or cord against contamination, and optionally, for bundling a plurality of the tube and/or cord with one another, comprising:
a flexible wall having opposite edges extending lengthwise between opposite ends, with said flexible wall having an open state and a closed state, wherein while the flexible wall is in the open state, one of said opposite edges is configured to be folded about the at least one tube and/or cord toward the other of said opposite sides to bring said flexible wall to the closed state to contain and shield the at least one tube and/or cord in an inner cavity bounded by an inner surface of said flexible wall; and
at least one closure member fixed to said flexible wall, said at least one closure member being configured to releasably fix said flexible wall in the closed state.

2. The disposable surgical tube and/or cord containment drape of claim 1, wherein said at least one closure member includes a first fastener portion and a second portion, said first fastener portion being configured for releasably fixed attachment to said second fastener portion.

3. The disposable surgical tube and/or cord containment drape of claim 2, wherein said first fastener portion is a hook portion fixed adjacent one of said opposite edges and said second fastener portion is a loop portion is fixed adjacent the other of said opposite edges.

4. The disposable surgical tube and/or cord containment drape of claim 1, further including at least one fixation member fixed to an outer surface of said flexible wall, wherein said at least one fixation member is configured to fix said flexible wall against movement relative to an external environment surface.

5. The disposable surgical tube and/or cord containment drape of claim 4, wherein said at least one fixation member includes a self-adhesive and a release film releasably covering said self-adhesive.

6. The disposable surgical tube and/or cord containment drape of claim 4, further including a pocket extending along said outer surface of said flexible wall, said at least one fixation member being fixed to a first side of said flexible wall, and said pocket extending along a second side of said flexible wall, said second side being opposite said first side.

7. The disposable surgical tube and/or cord containment drape of claim 6, wherein said pocket extends from an open end facing a first one of said opposite ends to a closed end facing a second one of said opposite ends.

8. The disposable surgical tube and/or cord containment drape of claim 7, further including an attachment member fixed to an inner surface of said pocket adjacent said open end, said attachment member being configured for attachment to a medical instrument apparatus.

9. A disposable surgical tube and/or cord containment drape for shielding at least one tube and/or cord against contamination, and optionally, for bundling a plurality of the tube and/or cord with one another, comprising:
a flexible wall having opposite edges extending lengthwise between opposite ends;
a fixation member extending adjacent one of the opposite edges, the fixation member including a self-adhesive for fixation to a surgical drape; and
at least one closure member adjacent the other of the opposite edges, the at least one closure member including a first fastener portion fixed to said flexible wall and a second fastener portion releasably attached to said first fastener portion, said second fastener portion having a third fastener portion facing away from said first fastener portion for fixation to the surgical drape.

10. The disposable surgical tube and/or cord containment drape of claim 9, wherein the first fastener portion is one of a hook portion or a loop portion, the second fastener portion is the other of the hook portion or the loop portion.

11. The disposable surgical tube and/or cord containment drape of claim 9, wherein the third fastener portion is a self-adhesive, and further including a first release film covering the self-adhesive of the fixation member and a second release film covering the self-adhesive of the third fastener portion, with the first release film being selectively removable to expose the self-adhesive of the fixation member for fixation to the surgical drape, and the second release film being selectively removable to expose the self-adhesive of the third fastener portion for fixation to the surgical drape.

12. The disposable surgical tube and/or cord containment drape of claim 9, further including a pocket extending along an outer surface of said flexible wall.

13. The disposable surgical tube and/or cord containment drape of claim 9, wherein said flexible wall is sterile.

14. A medical drape, comprising:
a drape layer; and
at least one surgical tube and/or cord containment drape fixed to said drape layer, said at least one surgical tube and/or cord containment drape configured to shield at least one tube and/or cord against contamination.

15. The medical drape of claim 14, wherein the at least one surgical tube and/or cord containment drape includes a flexible wall having opposite edges extending lengthwise between opposite ends, with said flexible wall having an open state and a closed state, wherein while said flexible wall is in the open state, a first of said opposite edges is attached to said drape layer, and a second of said opposite edges is configured to be folded over the at least one tube and/or cord into releasably attached relation to said drape layer to bring said flexible wall to the closed state to contain and shield the at least one tube and/or cord in an inner cavity bounded, at least in part, by an inner surface of said flexible wall.

16. The medical drape of claim 15, wherein said inner cavity is bounded along a first side by said inner surface of said flexible wall, and said inner cavity is bounded along a second side by said drape layer.

17. The medical drape of claim 16, further including a first fastener portion fixed to said flexible wall adjacent said second of said opposite edges and a second fastener portion fixed to said drape layer, said first fastener portion being configured for releasable attachment to said second fastener portion.

18. The medical drape of claim 17, wherein said first fastener portion is one of a hook portion or a loop portion and said second fastener portion is the other of the hook portion or the loop portion.

19. The medical drape of claim 15, wherein while the flexible wall is in the open state, one of said opposite edges is configured to be folded about the at least one tube and/or cord toward the other of said opposite edges to bring said flexible wall to the closed state to contain and shield the at least one tube and/or cord in an inner cavity bounded entirely by an inner surface of said flexible wall.

20. The medical drape of claim 19, further including at least one closure member including a first fastener portion fixed adjacent one of said opposite edges and a second fastener portion fixed adjacent the other of said opposite edges, said first fastener portion being configured for releasably fixed attachment to said second fastener portion to releasably fix said flexible wall in the closed state.

21. The medical drape of claim 15, wherein said at least one surgical tube and/or cord containment drape includes a pocket extending along an outer surface of said flexible wall.

22. The medical drape of claim 14, wherein the at least one surgical tube and/or cord containment drape includes a plurality of surgical tube and/or cord containment drapes attached to one another.

23. A method of constructing a disposable surgical tube and/or cord containment drape for shielding at least one tube and/or cord against contamination, and optionally, for bundling a plurality of the tube and/or cord with one another, comprising:
forming a flexible wall having opposite edges extending lengthwise along an axis between opposite ends, with the flexible wall being foldable along the axis from an open state to a closed state to bound an inner cavity by an inner surface of the flexible wall, with the inner cavity being sized for receipt of the surgical tube and/or cord therein; and
fixing at least one closure member to the flexible wall and configuring the at least one closure member to releasably fix the flexible wall in the closed state.

24. A method of constructing a disposable surgical tube and/or cord containment drape for shielding at least one tube and/or cord against contamination, and optionally, for bundling a plurality of the tube and/or cord with one another, comprising:
forming a flexible wall having opposite edges extending lengthwise between opposite ends;
fixing a fixation member extending adjacent one of the opposite edges, the fixation member including a self-adhesive for fixation to an external environment surface;
fixing a first fastener portion to the other of the opposite edges in spaced relation from the fixation member;
releasably attaching a second fastener portion to the first fastener portion, with the second fastener portion being provided having a self-adhesive facing away from the first fastener portion for fixation to the external environment surface.
